(19) United States
(12) Patent Application Publication
(10) Pub. No.: US 2023/0113650 A1
(43) Pub. Date: Apr. 13, 2023

(54) ENDOSCOPE, ENDOSCOPE SYSTEM AND STONE COLLECTION METHOD

(71) Applicant: OLYMPUS CORPORATION, Tokyo (JP)

(72) Inventor: Kazuya SASAGUCHI, Tokyo (JP)

(73) Assignee: OLYMPUS CORPORATION, Tokyo (JP)

(21) Appl. No.: 17/960,902

(22) Filed: Oct. 6, 2022

Related U.S. Application Data
(60) Provisional application No. 63/254,572, filed on Oct. 12, 2021.

(51) Int. Cl. A61B 1/018; A61B 1/00

(52) U.S. Cl. CPC A61B 1/018; A61B 1/0008

(57) ABSTRACT

An endoscope, comprising: an insertion portion insertable into an inside of a subject; an observation window provided in a distal end portion of the insertion portion; a liquid feeding channel extending through the distal end portion of the insertion portion; and at least one liquid feeding port. In operation, liquid exits the at least one liquid feeding port, and liquid exiting the at least one liquid feeding port is directed in a first direction away from the observation window and toward the inside of the subject.

### RELATED APPLICATION DATA

This application is based on and claims priority under 37 U.S.C. § 119 to U.S. Provisional Application No. 63/254,572 filed on Oct. 12, 2021, the entire contents of which are incorporated herein by reference.

### FIELD OF THE DISCLOSURE

The present disclosure relates to an endoscope that delivers liquid for perfusion of the inside of a subject, an endoscope system and a stone collection method.

### BACKGROUND

In recent years, various techniques in which when a stone present in an organ, such as a kidney, of a subject is removed, the stone is shattered into small fragments and the fragments are collected have been proposed.

One of methods used for shattering of a stone is a method in which laser light is generated from a laser apparatus. A ureteroscope is inserted into an organ of a subject, a laser probe connected to a laser apparatus is inserted through a treatment instrument channel of the ureteroscope and laser light is applied from the laser probe to a stone to shatter the stone.

For example, Japanese Patent Application Laid-Open Publication No. 2018-500986 describes a medical device including an image pickup device and an illumination device installed in a distal end surface of a tube, a suction port including an opening portion in the distal end surface of the tube, and a plurality of liquid feeding ports disposed in a side surface of the tube, wherein a laser probe for shattering a stone is inserted through the suction port. The stone shattered by application of laser light from the laser probe is collected from the suction port, together with liquid supplied from the liquid feeding ports.

### SUMMARY

An endoscope according to an aspect of the present disclosure includes: an insertion portion insertable into an inside of a subject; an observation window provided in a distal end portion of the insertion portion; a liquid feeding channel extending through the distal end portion of the insertion portion; and at least one liquid feeding port. In operation, liquid exits the at least one liquid feeding port, and wherein liquid exiting the at least one liquid feeding port is directed in a first direction away from the observation window and toward the inside of the subject.

An endoscope system according to an aspect of the present disclosure includes: the endoscope described above; and a liquid feeding/suction apparatus operably connected to the endoscope. The endoscope further includes a suction channel, and wherein the liquid feeding/suction apparatus includes a liquid feeding pump configured to feed the liquid to the at least one liquid feeding port and a suction pump configured to suction up the liquid via the suction channel.

A stone collection method according to the present disclosure includes: shattering a stone inside a subject via a probe; conveying liquid via a liquid feeding channel of an endoscope; delivering a first portion of the liquid via at least one liquid feeding port of the endoscope in a first direction toward an observation window provided in a distal end portion of an insertion portion of the endoscope; delivering a second portion of the liquid via the at least one liquid feeding port of the endoscope in a second direction toward the inside of the subject from a second liquid feeding port of the liquid feeding channel; and suctioning up the liquid and the shattered stone via a suction channel of the endoscope.

### BRIEF DESCRIPTION OF THE DRAWINGS

FIG. 1 is a diagram illustrating an example configuration of an endoscope system according to a first embodiment of the present disclosure.

FIG. 2 is a diagram illustrating an example configuration of an endoscope according to the first embodiment.

FIG. 3 is a perspective diagram illustrating a configuration of a distal end portion of the endoscope, through which a liquid feeding tube and a laser probe are inserted, in the first embodiment.

FIG. 4 is a diagram illustrating a distal end surface of the distal end portion of the endoscope, through which the liquid feeding tube and the laser probe are inserted, in the first embodiment.

FIG. 5 is a sectional view in an axial direction, the sectional view illustrating a first liquid feeding port provided in a tube distal end part of the liquid feeding tube in the first embodiment.

FIG. 6 is a sectional view in the axial direction, the sectional view illustrating a second liquid feeding port provided in the tube distal end part of the liquid feeding tube in the first embodiment.

FIG. 7 is a sectional view perpendicular to the axial direction, the sectional view illustrating the first liquid feeding port and the second liquid feeding port provided in the tube distal end part of the liquid feeding tube in the first embodiment.

FIG. 8 is a perspective diagram illustrating a manner in which liquid is fed to the inside of a subject from the first liquid feeding port and the second liquid feeding port of the liquid feeding tube and liquid is collected from a treatment instrument channel that doubles as a suction channel in the first embodiment.

FIG. 9 is a diagram illustrating a manner in which a renal calyx of a kidney is perfused with liquid in the first embodiment.

FIG. 10 is a flowchart illustrating a stone collection method using the endoscope system according to the first embodiment.

FIG. 11 is a perspective diagram illustrating a configuration of a distal end portion of an endoscope, through which a laser probe is inserted, in a second embodiment of the present disclosure.

FIG. 12 is a diagram illustrating a distal end surface of the distal end portion of the endoscope, through which the laser probe is inserted, together with flows of liquid, in the second embodiment.

FIG. 13 is a sectional view along 13A-13A in FIG. 12, the sectional view illustrating a manner in which liquid flows from a liquid feeding channel to an observation window through an extending shape portion in the second embodiment.

FIG. 14 is a sectional view illustrating a manner in which liquid flows from a liquid feeding channel to an observation window through a projecting shape portion in a third embodiment of the present disclosure.

FIG. 15 is a diagram indicating a relationship between respective lengths of projection, in an axial direction of an insertion portion, of a channel opening of the liquid feeding channel and the observation window in the third embodiment.

FIG. 16 is a diagram illustrating a distal end surface of a distal end portion of an endoscope, through which a laser probe is inserted, together with flows of liquid, in the third embodiment.

FIG. 17 is a sectional view illustrating a configuration of a part around a channel opening of a liquid feeding channel in a fourth embodiment of the present disclosure.

FIG. 18 is a diagram illustrating a distal end surface of a distal end portion of an endoscope, through which a laser probe is inserted, together with flows of liquid, in the fourth embodiment.

FIG. 19 is a diagram illustrating a manner in which liquid is delivered from the channel opening of the liquid feeding channel to an observation window as viewed from a lateral side in the fourth embodiment.

FIG. 20 is a sectional view illustrating a configuration of a part around a channel opening of a liquid feeding channel in a fifth embodiment of the present disclosure.

### DETAILED DESCRIPTION

Generally, applying laser light to a stone to shatter the stone causes vaporization of water inside the stone, resulting in generation of gas bubbles. The generated gas bubbles move toward a suction port together with liquid. However, in the technique described in Japanese Patent Application Laid-Open Publication No. 2018-500986, because of the suction port being disposed in the distal end surface of the working tube, in which an image pickup device and an illumination device are also installed, the gas bubbles that have moved together with the liquid may adhere to outer surfaces (e.g., lens surfaces) of the image pickup device and the illumination device. In particular, adherence of gas bubbles to the lens surface of the image pickup device results in degradation in quality of a picked-up image. Conventionally, such a problem of observation performance degradation due to adherence of gas bubbles to a surface of an observation system when a stone is collected has received little attention.

Each of embodiments described below enables provision of an endoscope, an endoscope system and a stone collection method that prevent observation performance degradation when liquid is collected together with a shattered stone.

Embodiments of the present disclosure will be described below with reference to the drawings. However, the present disclosure is not limited by the below-described embodiments.

Note that in the drawings, elements that are identical or correspond to each other are appropriately provided with a same reference numeral. Also, it should be noted that the drawings are schematic ones, and for simplicity of illustration, e.g., relationships in length among respective elements, ratios in length among the respective elements and numbers and quantities of the respective elements in one drawing may be different from actual ones. Furthermore, parts that are different in relationship in length or ratio depending on a plurality of drawings may be included.

### First Embodiment

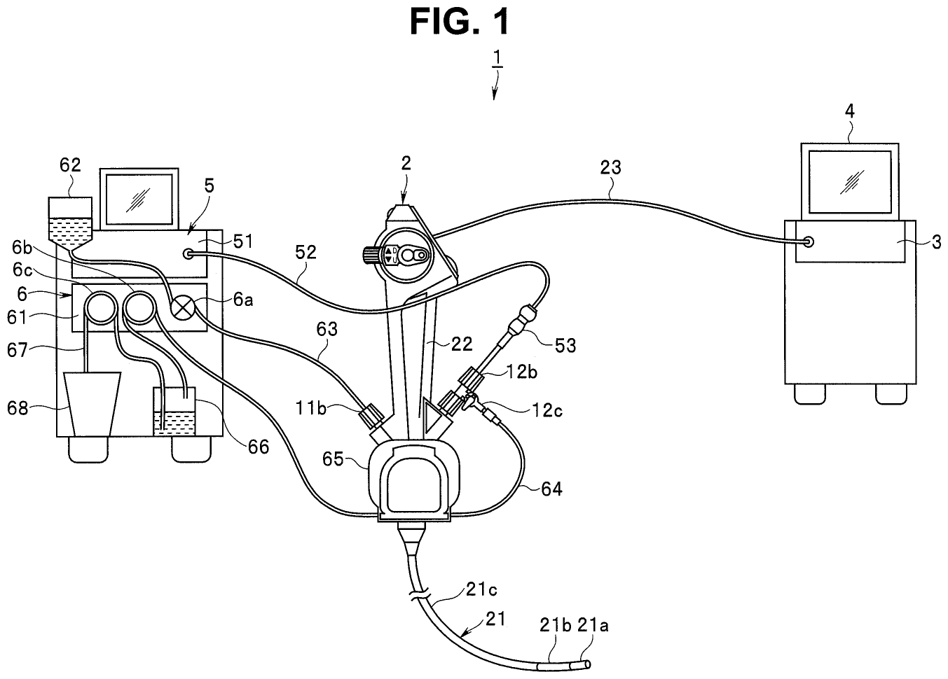

FIGS. 1 to 10 illustrates a first embodiment of the present disclosure and FIG. 1 is a diagram illustrating an example configuration of an endoscope system 1.

As illustrated in FIG. 1, the endoscope system 1 of the present embodiment includes an endoscope 2, an endoscope control apparatus 3, a monitor 4, a laser system 5 (lithotripsy apparatus) and a pump system 6 (liquid feeding/suction apparatus).

The endoscope 2 is a device configured to allow observation and procedure of a subject. The endoscope 2 includes an elongated insertion portion 21 that is inserted into the inside of a subject, an operation portion 22 provided in such a manner as to be continuous with the proximal end side of the insertion portion 21 and a universal cable 23 extending from the operation portion 22. Note that the subject to which the insertion portion 21 is inserted is assumed to be a living organism such as a human being or an animal but is not limited to this example and may be a non-living material such as a machine or an architecture.

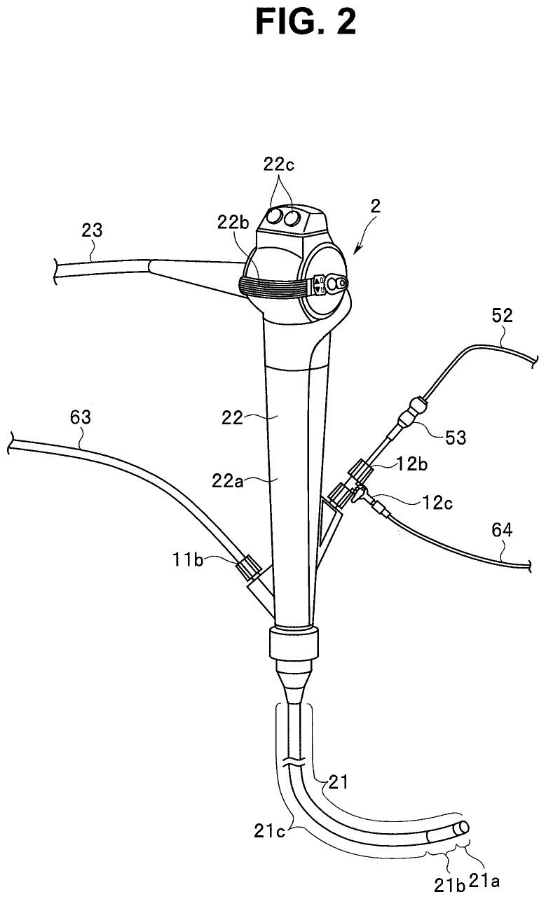

FIG. 2 is a diagram illustrating an example configuration of the endoscope 2.

As illustrated in FIG. 2, the insertion portion 21 includes a distal end portion 21a, a bending portion 21b and a tubular portion 21c in the order mentioned above from a distal end to a proximal end.

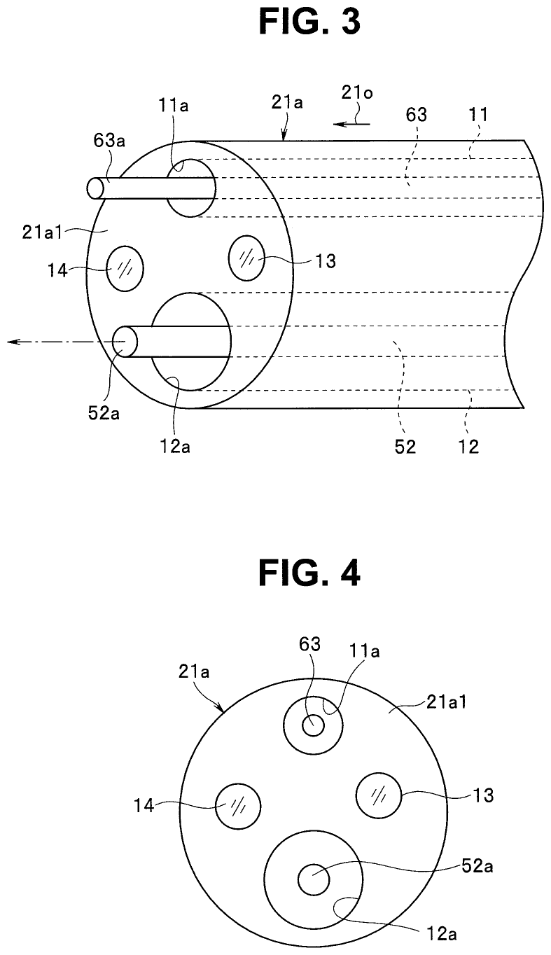

The distal end portion 21a includes an observation system and an illumination system. The observation system includes an observation window 13 (see, e.g., FIG. 3) and an objective optical system, and if the endoscope 2 is an electronic endoscope, further includes an image pickup device, and if the endoscope 2 is an optical endoscope, further includes an image guide. The below description will be provided on the assumption that the endoscope 2 is an electronic endoscope. The image pickup device includes, for example, an image sensor of, e.g., a CMOS or a CCD, and a signal wire is connected to the image pickup device. Note that the observation window 13 may doubles as a distal end lens of the objective optical system.

The illumination system includes, for example, an illumination window 14 (see, e.g., FIG. 3) that doubles as an illumination optical system, and a light guide. The light guide is configured as, for example, a fiber bundle formed by bundling optical fibers. The signal wire connected to the image pickup device, and the light guide are installed inside each of the insertion portion 21, the operation portion 22 and the universal cable 23 and is connected to the endoscope control apparatus 3.

FIG. 3 is a perspective diagram illustrating a configuration of the distal end portion 21a of the endoscope 2, through which a liquid feeding tube 63 and a laser probe 52 are inserted. FIG. 4 is a diagram illustrating a distal end surface 21a1 of the distal end portion 21a of the insertion portion 21 of the endoscope 2.

As illustrated in FIGS. 3 and 4, a liquid feeding channel 11 configured to convey liquid, and a treatment instrument channel 12 are inserted through the inside of the insertion portion 21. In the distal end portion 21a, a channel opening 11a on the distal end side of the liquid feeding channel 11 and a channel opening 12a (treatment instrument opening) on the distal end side of the treatment instrument channel 12 are provided.

The bending portion 21b is provided in such a manner as to be continuous with the proximal end side of the distal end portion 21a and is configured in such a manner as to be bendable, for example, in two directions or four, up, down, left and right, directions. When the bending portion 21b is bent, a direction of the distal end portion 21a changes, and thus, a direction of observation via the observation system and a direction of application of illuminating light by the illumination system changes. The bending portion 21b is also bent for enhancement in insertability of the insertion portion 21 inside the subject.

The tubular portion 21c is a tubular part joining a proximal end of the bending portion 21b and a distal end of the operation portion 22. The tubular portion 21c may have a rigid form that prevents the insertion portion 21 from bowing or may have a flexible form that allows the insertion portion 21 to bow according to a shape of the subject. An endoscope including an insertion portion having a rigid form is generally called a rigid endoscope and an endoscope including an insertion portion having a flexible form is generally called a flexible endoscope. For example, a rigid endoscope and a flexible endoscope in the medical field are defined in ISO8600-1:2015.

The operation portion 22 is a part provided in such a manner as to be continuous with the proximal end side of the insertion portion 21, the part being configured to be held by a hand to perform various operations for the endoscope 2. The operation portion 22 includes, for example, a grasping portion 22a, a bending operation lever 22b, a plurality of operation buttons 22c, a channel opening 11b on the proximal end side of the liquid feeding channel 11, a channel opening 12b on the proximal end side of the treatment instrument channel 12, and a suction tube attachment bracket 12c of the treatment instrument channel 12.

The grasping portion 22a is a part of the endoscope 2, the part being grasped by an operator with his/her hand.

The bending operation lever 22b is an operation device for performing an operation to bend the bending portion 21b, using, for example, the thumb of the hand grasping the grasping portion 22a.

The plurality of operation buttons 22c include, for example, a liquid feeding button and a suction button. The liquid feeding button is an operation button for feeding liquid to the distal end portion 21a side via the liquid feeding channel 11. The suction button is an operation button for performing suction from the distal end portion 21a side via the treatment instrument channel 12 that doubles as the suction channel. Also, the plurality of operation buttons 22c may include, for example, a button switch for performing an operation for image pickup (e.g., a release operation).

The channel opening 11b on the proximal end side of the liquid feeding channel 11 is provided in a side surface on the distal end side of the grasping portion 22a. The liquid feeding tube 63 is inserted into the liquid feeding channel 11 from the channel opening 11b.

The channel opening 12b on the proximal end side of the treatment instrument channel 12 is provided in another side surface on the distal end side of the grasping portion 22a. The laser probe 52 is inserted into the channel opening 12b using a protective tube 53. The protective tube 53 prevents bending of a laser fiber included in the laser probe 52. The treatment instrument channel 12 is used to allow insertion of various treatment instruments. Therefore, instead of the laser probe 52, a treatment instrument such as a forceps may be inserted through the inside of the treatment instrument channel 12.

Also, the treatment instrument channel 12 doubles as the suction channel configured to suck up liquid inside the subject together with a shattered stone. A first suction tube 64 is connected to the suction tube attachment bracket 12c provided in the vicinity of the channel opening 12b on the proximal end side of the treatment instrument channel 12.

The universal cable 23 extends from a side surface on the proximal end side of the operation portion 22 and is connected to the endoscope control apparatus 3.

The endoscope control apparatus 3, which serves as both an image processing apparatus and a light source apparatus, controls the endoscope 2, processes an image pickup signal acquired from the endoscope 2 and supplies illuminating light to the endoscope 2.

The endoscope control apparatus 3 includes a plurality of light sources configured to emit illuminating light such as white light or special observation light, a light source control circuit configured to control the light sources, and an optical system configured to collect light emitted from the light sources into an input end of the light guide. For each light source, any type of light source such as an LED (light-emitting diode) light source, a laser light source, a xenon light source or a halogen light source may be used as long as the light source is a device configured to emit illuminating light, or a plurality of types of light sources may be combined.

The image processing apparatus and the light source apparatus may be configured as separate apparatuses. Also, instead of the configuration in which illuminating light from the endoscope control apparatus 3 is supplied to the endoscope 2, a configuration in which a light-emitting element disposed in the distal end portion 21a emits illuminating light may be employed.

Each of parts in which light source control, image processing and various types of control are performed in the endoscope control apparatus 3 may be configured in such a manner that all or some of functions of the part is implemented by, for example, a processor such as an ASIC (application-specific integrated circuit) including a CPU (central processing unit) or an FPGA (field-programmable gate array) reading, for example, a computer program (software) stored in a computer-readable non-transitory storage device such as a ROM (read-only Memory) (or an HDD (hard disk drive), an SSD (solid-state drive) or a disk-like recording medium), loading the computer program onto a RAM (random access memory) and executing the computer program. However, the configuration of the endoscope control apparatus 3 is not limited to this example, and, for example, the endoscope control apparatus 3 may be configured in such a manner that all or some of the functions of each of the parts are implemented by a dedicated electronic circuit.

The endoscope 2 and the endoscope control apparatus 3 are electrically and optically connected by connecting the universal cable 23 to a connector receiver of the endoscope control apparatus 3.

Illuminating light emitted from the endoscope control apparatus 3 that doubles as the light source apparatus is transmitted by the light guide and applied to the subject from the illumination window 14 of the distal end portion 21a. Return light from the subject to which the illuminating light has been applied travels through the observation window 13 and forms an image in the image pickup device via the objective optical system.

The endoscope control apparatus 3 transmits a drive signal and electric power to the image pickup device. The image pickup device picks up an optical image of the subject in response to the drive signal and generates an image pickup signal. Image pickup by the image pickup device is continuously performed, for example, on a frame-by-frame basis and an image pickup signal for a plurality of frames of a movie are generated. The image pickup signal is transmitted to the endoscope control apparatus 3 via the signal wire.

The endoscope control apparatus 3 receives the image pickup signal obtained by the image pickup device and performs various types of image processing such as demosaicking, noise correction, color correction, contrast correction and/or gamma correction to generate a displayable image signal. The endoscope control apparatus 3 may superimpose various types of information such as text information and/or guide information on the image signal.

The image signal generated by the endoscope control apparatus 3 is outputted to the monitor 4. The monitor 4, which is a display device, receives the image signal from the endoscope control apparatus 3 and displays an endoscopic image.

The laser system 5 includes a laser apparatus body 51 and the laser probe 52. The laser apparatus body 51 generates laser light that shatters a stone present in, e.g., a kidney, a ureter, a bladder or a urethra in a urinary tract (urinary tract stone). The laser probe 52 includes the laser fiber configured to transmit laser light. A probe distal end 52a of the laser probe 52 is made to project from the channel opening 12a on the distal end side of the treatment instrument channel 12 and laser light is generated by the laser apparatus body 51. Then, the laser light is applied from the probe distal end 52a illustrated in FIGS. 3 and 4 to the stone inside the subject.

The pump system 6 includes a pump body 61 including a liquid feeding pump 6a, a first suction pump 6b and a second suction pump 6c, a liquid feeding tank 62, a liquid feeding tube 63, a first suction tube 64, a first filter 65, a second filter 66, a second suction tube 67 and a waste tank 68.

The liquid feeding tank 62 stores liquid to be fed to the inside of the subject. The liquid stored in the liquid feeding tank 62 is, for example, saline.

The liquid feeding tank 62 is connected to the liquid feeding pump 6a via the liquid feeding tube 63, and a part of the liquid feeding tube 63, the part being on the distal end side relative to the liquid feeding pump 6a, is inserted into the liquid feeding channel 11 from the channel opening 11b on the proximal end side.

As illustrated in FIG. 3, a tube distal end part 63a of the liquid feeding tube 63 inserted through the liquid feeding channel 11 projects from the channel opening 11a on the distal end side of the liquid feeding channel 11. When the liquid feeding pump 6a operates, the liquid inside the liquid feeding tank 62 is fed through the inside of the liquid feeding tube 63 and delivered to the inside of the subject from the tube distal end part 63a of the liquid feeding tube 63 inserted through the inside of the liquid feeding channel 11.

Therefore, the liquid feeding channel 11 of the present embodiment conveys the liquid via the liquid feeding tube 63 inserted through the inside of the liquid feeding channel 11.

When the first suction tube 64 is connected to the suction tube attachment bracket 12c, the first suction tube 64 is brought into communication with the inside of the treatment instrument channel 12. Even when the laser probe 52 is inserted through the inside of the treatment instrument channel 12, suction via the first suction tube 64 is possible because of there being a gap between the laser probe 52 and the treatment instrument channel 12.

The first suction tube 64 is connected to the second filter 66 via the first filter 65 and the first suction pump 6b. Each of the first filter 65 and the second filter 66 is an instrument configured to filter a stone and a mucous membrane sucked up from the inside of the subject. Of the filters, for example, the first filter 65 is used for collecting a stone. The first filter 65 is attached to, for example, the distal end side of the grasping portion 22a in the operation portion 22 of the endoscope 2 (however, the disposition of the first filter 65 is not limited to the above disposition).

An end of the second suction tube 67 is connected to the second filter 66 and the other end of the second suction tube 67 is connected to the waste tank 68 via the second suction pump 6c.

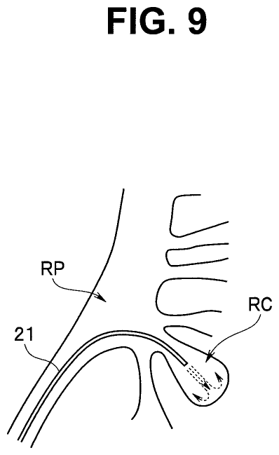

The first suction pump 6b and the second suction pump 6c operate in conjunction with each other and suck up liquid inside the subject from the treatment instrument channel 12 that doubles as the suction channel. At this time, by the liquid feeding pump 6a also operating in conjunction with the first suction pump 6b and the second suction pump 6c, feeding of liquid into the subject via the liquid feeding tube 63 and suction of liquid inside the subject via the treatment instrument channel 12 that doubles as the suction channel are performed simultaneously. A circulation flow of the liquid that has perfused the inside of the subject occurs and a shattered stone is carried by the flow (see FIG. 9), enhancing efficiency of collection of the stone.

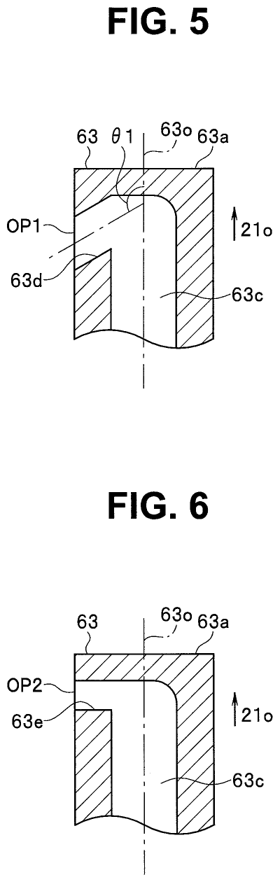

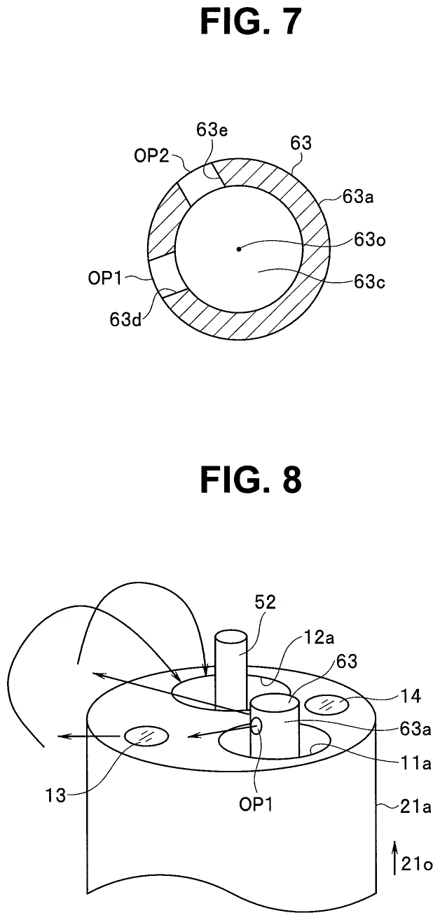

FIG. 5 is a sectional view in an axial direction 21o, the sectional view illustrating a first liquid feeding port OP1 provided in the tube distal end part 63a of the liquid feeding tube 63. FIG. 6 is a sectional view in the axial direction 21o, the sectional view illustrating a second liquid feeding port OP2 provided in the tube distal end part 63a of the liquid feeding tube 63. FIG. 7 is a sectional view perpendicular to the axial direction 21o, the sectional view illustrating the first liquid feeding port OP1 and the second liquid feeding port OP2 provided in the tube distal end part 63a of the liquid feeding tube 63. FIG. 8 is a perspective diagram illustrating a manner in which liquid is fed to the inside of the subject from the first liquid feeding port OP1 and the second liquid feeding port OP2 of the liquid feeding tube 63 and collected from the treatment instrument channel 12 that doubles as the suction channel. FIG. 9 is a diagram illustrating a manner in which the inside of a renal calyx RC of a kidney is perfused with liquid. As can be seen with reference to FIG. 7, FIGS. 5 and 6 are sectional views in the axial direction 21o at different angles around a center axis 63o of the liquid feeding tube 63.

The liquid feeding tube 63 includes a liquid feeding conduit 63c inside. In the conduit 63c in the tube distal end part 63a of the liquid feeding tube 63, a first branch channel 63d, which is illustrated in FIG. 5, and a second branch channel 63e, which is illustrated in FIG. 6, are provided. The first branch channel 63d is turned back at an angle θ1 that is more than 90 degrees (that is, θ1>90° to the center axis 63o of the liquid feeding tube 63. The second branch channel 63e may intersect with the center axis 63o at an appropriate angle.

The first liquid feeding port OP1 is an opening formed in a side surface of the tube distal end part 63a. The first liquid feeding port OP1 communicates with the conduit 63c via the first branch channel 63d and delivers liquid conveyed by the conduit 63c, in a direction of the observation window 13. The liquid delivered via the first liquid feeding port OP1 removes a substance adhering to the observation window 13.

The second liquid feeding port OP2 is an opening formed in another side surface of the tube distal end part 63a independently from the first liquid feeding port OP1. The second liquid feeding port OP2 communicates with the conduit 63c via the second branch channel 63e, and delivers the liquid conveyed by the conduit 63c, in a direction (direction of the inside of the subject) that is different from the direction of the observation window 13. The inside of the subject is perfused with the liquid delivered from the second liquid feeding port OP2.

The treatment instrument channel 12 that doubles as the suction channel sucks up the liquid together with a shattered stone, the liquid being delivered from the first liquid feeding port OP1 and the second liquid feeding port OP2, the inside of the subject being perfused with the liquid. As illustrated in FIG. 9, if a circulating flow along the inner wall of an organ can be formed in, e.g., a renal pelvis RP or a renal calyx RC, efficiency of stone collection is enhanced. FIG. 8 illustrates a manner in which liquid delivered from the first liquid feeding port OP1 circulates through the observation window 13 and returns to the channel opening 12a and liquid delivered from the second liquid feeding port OP2 circulates and returns to the channel opening 12a.

For reduction in diameter of the insertion portion 21 of the endoscope 2, as described above, the treatment instrument channel 12 can be used as the suction channel. However, the treatment instrument channel 12 is not limited to this example and a treatment instrument channel 12 and a suction channel may separately be provided.

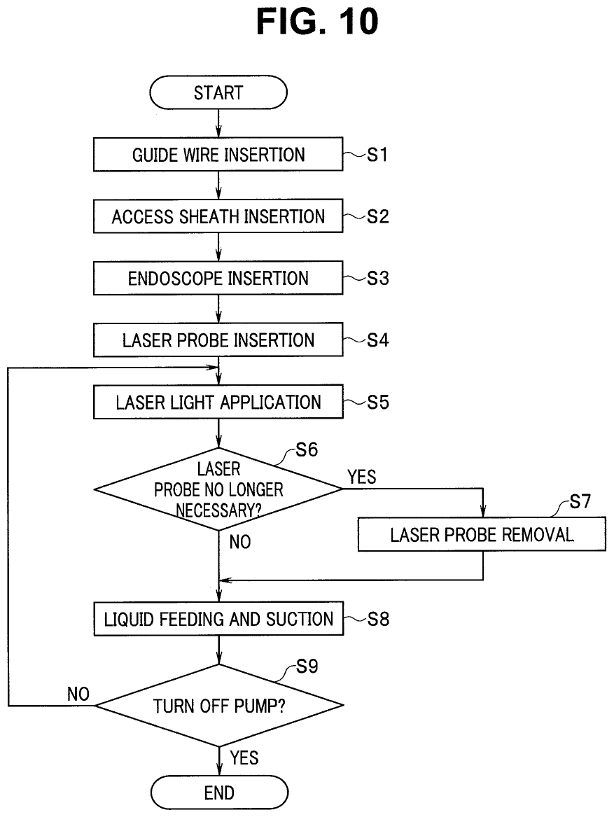

FIG. 10 is a flowchart illustrating a stone collection method using the endoscope system 1.

When a procedure is started, first, for example, a surgeon inserts a guide wire into a subject and makes a distal end of the guide wire reach the inside of an organ such as a kidney (step S1).

Next, the surgeon inserts an access sheath along the guide wire (step S2). After the insertion of the access sheath, the surgeon pulls the guide wire out.

Subsequently, the surgeon inserts the insertion portion 21 of the endoscope 2 to the inside of the subject through the access sheath (step S3). The surgeon looks for a stone to be collected, while observing an endoscopic image acquired via the observation system of the endoscope 2 on the monitor 4.

When the stone is observed, the surgeon inserts the laser probe 52 through the treatment instrument channel 12, makes the laser probe 52 project from the channel opening 12a and makes the probe distal end 52a of the laser probe 52 face the stone (step S4).

Then, laser light is generated by the laser system 5 and the laser light is applied to the stone inside the subject from the probe distal end 52a (step S5). When the laser light is applied to the stone, water inside the stone evaporates and rapidly swells and the stone is shattered by pressure of the evaporated water into a plurality of smaller fragments. At this time, gas bubbles are generated by evaporation of water in the stone and moisture present on an optical path on which the laser light travels.

Subsequently, the surgeon determines whether or not the laser probe 52 is no longer necessary, that is, whether or not all of one or more target stones have been subjected to shattering processing (step S6), and if the surgeon determines that the laser probe 52 is no longer necessary, the surgeon removes the laser probe 52 from the endoscope 2 (step S7).

In step S6, if the surgeon determines that the laser probe 52 is still necessary or if the removal in step S7 is finished, feeding of liquid to the inside of the subject via the liquid feeding tube 63 inserted through the liquid feeding channel 11 and suction of liquid inside the subject via the treatment instrument channel 12 that doubles as the suction channel are performed simultaneously (step S8). Therefore, the liquid feeding and the liquid suction are performed both during the laser probe 52 being inserted through the treatment instrument channel 12 and after the removal of the laser probe 52 from the treatment instrument channel 12, that is, may be performed after shattering of the stone or may be performed simultaneously with shattering of the stones.

Consequently, a flow in which the liquid is delivered from the first liquid feeding port OP1 and the second liquid feeding port OP2, circulates inside the subject and returns to the channel opening 12a occurs, and the shattered stones are sucked up from the channel opening 12a together with the liquid.

At this time, since the liquid is delivered in the direction of the observation window 13 from the first liquid feeding port OP1, even if gas bubbles, shattered stones and/or tissues such as parts of exfoliated mucous membranes adhere to the observation window 13, such gas bubbles, shattered stones and/or tissues can be removed by the liquid flow.

Also, the liquid delivered from the first liquid feeding port OP1 generates a flow that radially covers the observation window 13, enabling preventing the liquid that has returned after circulation (that is, the liquid carrying the gas bubbles and the shattered stones) from passing over the observation window 13 again and thus, enabling preventing the gas bubbles and/or the shattered stones from newly adhering to the observation window 13.

Furthermore, the liquid is delivered from the second liquid feeding port OP2 in a direction (direction of the inside of the subject) that is different from the direction of the observation window 13, enabling making a route of a flow by which gas bubbles and/or shattered stones are mainly carried different from the route through the observation window 13, that is, enabling reducing gas bubbles and shattered stones flowing toward the observation window 13.

When a flow rate of liquid is high, shattered stones scatter, which may result in decrease in efficiency of collection of the stones. On the other hand, in the present embodiment, the liquid is fed from the first liquid feeding port OP1 and the second liquid feeding port OP2, which are a plurality of liquid feeding ports, enabling reducing a flow rate of the liquid delivered from each one liquid feeding port and thus enabling prevention of scattering of the stone, which enhances collection efficiency.

The surgeon finishes collection of the stones and determines whether or not to turn off the pump system 6 (step S9), and if the surgeon determines not to turn off the pump system 6, the surgeon returns the procedure to step S5 and performs application of laser light as necessary, and continues to perform liquid feeding and suction in step S8. Where the laser probe 52 has been removed in step S7, the surgeon returns the procedure from step S9 to step S8 and perform liquid feeding and suction only.

On the other hand, in step S9, if the surgeon determines to turn off the pump system 6, the surgeon stops the pump system 6.

Subsequently, the surgeon may remove the endoscope 2 from the subject and end the procedure or may move the endoscope 2 to another renal calyx RC and further look for a stone. After completion of collection of all of stones from the subject, the surgeon ends the processing.

According to the first embodiment, liquid is delivered in the direction of the observation window 13 from the first liquid feeding port OP1 and liquid is delivered in a direction (direction of the inside of the subject) that is different from the direction of the observation window 13 from the second liquid feeding port OP2, enabling preventing deterioration in observation performance when the liquid is collected together with a shattered stone.

Since the first liquid feeding port OP1 delivers liquid that removes a substance adhering to the observation window 13, optical performance of the observation window 13 is maintained.

Liquid is conveyed by the liquid feeding tube 63 inserted through the inside of the liquid feeding channel 11, the first liquid feeding port OP1 is formed in a side surface of the tube distal end part 63a and the second liquid feeding port OP2 is formed in another side surface of the tube distal end part 63a, enabling the liquid to be delivered in a plurality of different directions with no need to form the distal end side of the liquid feeding channel 11 into a particular shape.

A liquid flow that obliquely passes toward a surface of the liquid observation window 13 can favorably be formed by folding the first branch channel 63d of the liquid feeding tube 63 back at an angle θ1 that is more than 90 degrees to the center axis 63o of the liquid feeding tube 63.

Application of laser light from the laser probe 52 inserted through the treatment instrument channel 12 to a stone enables correctly shattering the stone under observation via the endoscope 2.

The treatment instrument channel 12 doubling as the suction channel enables reduction in diameter of the insertion portion 21.

### Second Embodiment

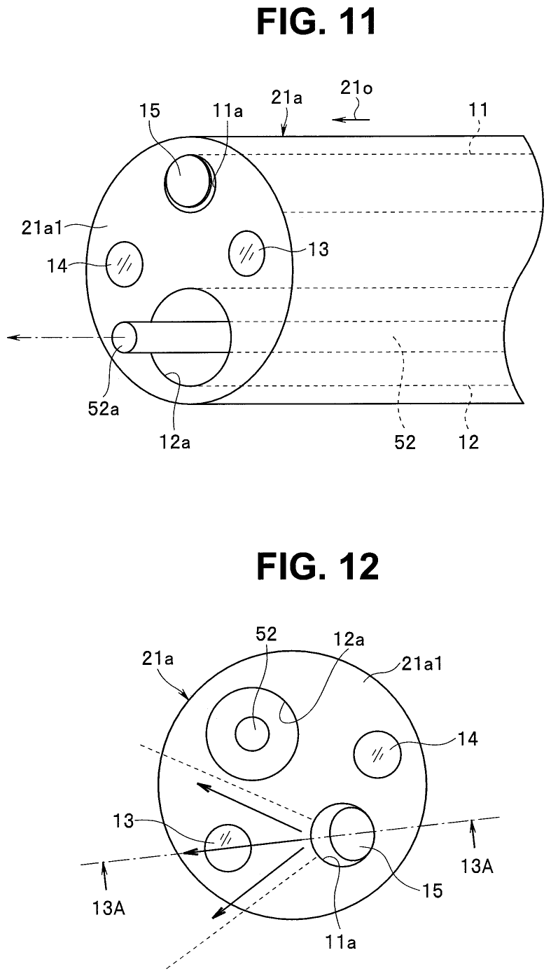

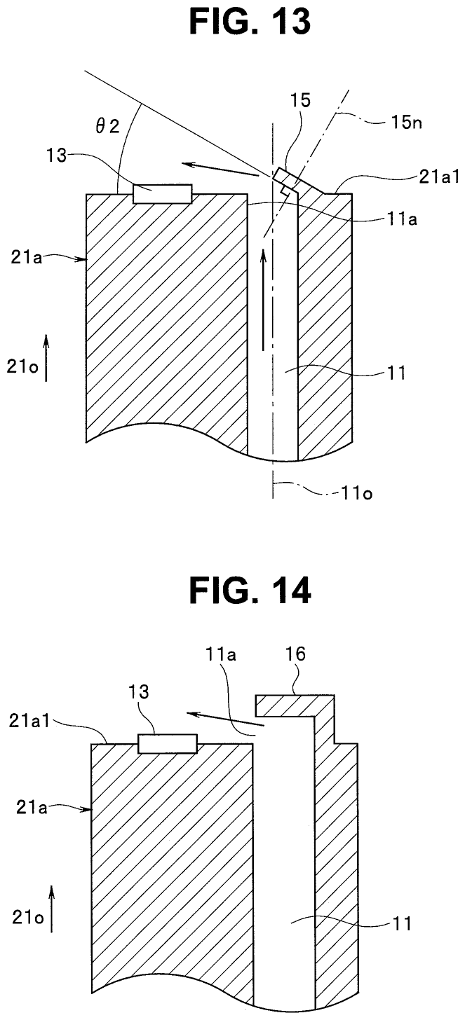

FIGS. 11 to 13 illustrate a second embodiment of the present disclosure. FIG. 11 is a perspective diagram illustrating a configuration of a distal end portion 21a of an endoscope 2, through which a laser probe 52 is inserted. FIG. 12 is a diagram illustrating a distal end surface 21a1 of the distal end portion 21a of the endoscope 2, through which the laser probe 52 is inserted, together with flows of liquid. FIG. 13 is a sectional view along 13A-13A in FIG. 12, the sectional view illustrating a manner in which liquid flows from a liquid feeding channel 11 to an observation window 13 through an extending shape portion 15. The extending shape portion 15 may include a deflector. In the second embodiment, parts that are similar to the parts in the first embodiment are provided with reference numerals that are the same as the reference numerals in the first embodiment and description of such parts will appropriately be omitted and description will be provided mainly on differences.

In the present embodiment, a liquid feeding tube 63 is not inserted through the inside of the liquid feeding channel 11 but is connected to a channel opening 11b on the proximal end side of a liquid feeding channel 11. Liquid fed from a liquid feeding tank 62 via the liquid feeding tube 63 is further fed from the channel opening 11b on the proximal end side to a channel opening 11a on the distal end side via the liquid feeding channel 11.

In the channel opening 11a of the liquid feeding channel 11, an extending shape portion 15 extending to the channel opening 1 lain such a manner as to cover a part of the channel opening 11a is provided. The extending shape portion 15 may be configured integrally with a distal end portion 21a of an endoscope 2 or may be configured separately from a distal end portion 21a of an endoscope 2 and attached to the distal end portion 21a.

The extending shape portion 15 is formed in, for example, a flat plate-like shape and is connected to an edge of the channel opening 11a at a position on the opposite side of the channel opening 11a from an observation window 13. Also, a side of the extending shape portion 15, the side facing the observation window 13, is raised at an appropriate angle θ2 (see FIG. 13) from the distal end surface 21a1 of the distal end portion 21a of the endoscope 2.

The angle θ2 of the raising of a flat plate-like principal surface of the extending shape portion 15 can be an angle that is more than 0 degrees and no more than 45 degrees. However, the angle θ2 is not limited to this example and may be any angle as long as the angle enables a liquid flow to be delivered to the observation window 13 side at an adequate flow rate.

FIG. 13 illustrates a manner in which a plane including a center line 11o of the liquid feeding channel 11 and a normal 15n to the principal surface, the normal 15n intersecting with the center line 11o (section along 13A-13A in FIG. 12) intersects with the observation window 13.

The liquid feeding channel 11 conveys liquid in an axial direction 21o of an insertion portion 21 (direction of the center line 11o of the liquid feeding channel 11). The extending shape portion 15 changes the direction of conveyance of the liquid from the axial direction 21o to a surface direction along the distal end surface 21a1 of the distal end portion 21a. Consequently, the channel opening 11a delivers the liquid in the surface direction.

A part of the channel opening 11a from which the extending shape portion 15 extends functions as a first liquid feeding port OP1 and another part of the channel opening 11a functions as a second liquid feeding port OP2. Therefore, a part of the liquid delivered from the channel opening 11a flows toward the observation window 13 as a liquid flow that prevents adherence of gas bubbles and another part of the liquid flows in a direction (direction of the inside of a subject) that is different from the direction of the observation window 13 as a liquid flow for perfusion (see arrows in FIG. 12). As illustrated in FIG. 12, the extending shape portion 15 is configured to allow a liquid flow to spread radially.

The extending shape portion 15 covers a part of the channel opening 11a with the observation window 13 side raised, preventing liquid returned after circulation inside the subject (that is, liquid that carries gas bubbles and a shattered stone) from merging with liquid delivered from the channel opening 11a and flowing toward the observation window 13 side.

Such second embodiment as above exerts effects that are substantially similar to the effects of the above-described first embodiment, but unlike the first embodiment, eliminates the step of making adjustment to provide a proper length of projection of a liquid feeding tube 63 from the channel opening 11a in the case where the liquid feeding tube 63 projects from the channel opening 11a, and eliminates the step of adjusting a rotational direction of a liquid feeding tube 63 in such a manner that the first liquid feeding port OP1 faces the observation window 13. In the present embodiment, the liquid feeding tube 63 is simply connected to the channel opening 11b and thus, handling of the liquid feeding tube 63 is easy.

Also, since the channel opening 11a serves as both the first liquid feeding port OP1 and the second liquid feeding port OP2, it is not necessary to provide a plurality of independent openings, and thus, the configuration is simplified.

Furthermore, setting the angle θ2 of the raising of the flat plate-like principal surface of the extending shape portion 15 as an angle that is more than 0 degrees and no more than 45 degrees enables preventing a decrease in collection efficiency due to scattering of a shattered stone and gas bubbles.

The plane in FIG. 13, which is illustrated as a section along 13A-13A in FIG. 12, intersecting with the observation window 13 enables efficient generation of a liquid flow toward the observation window 13.

### Third Embodiment

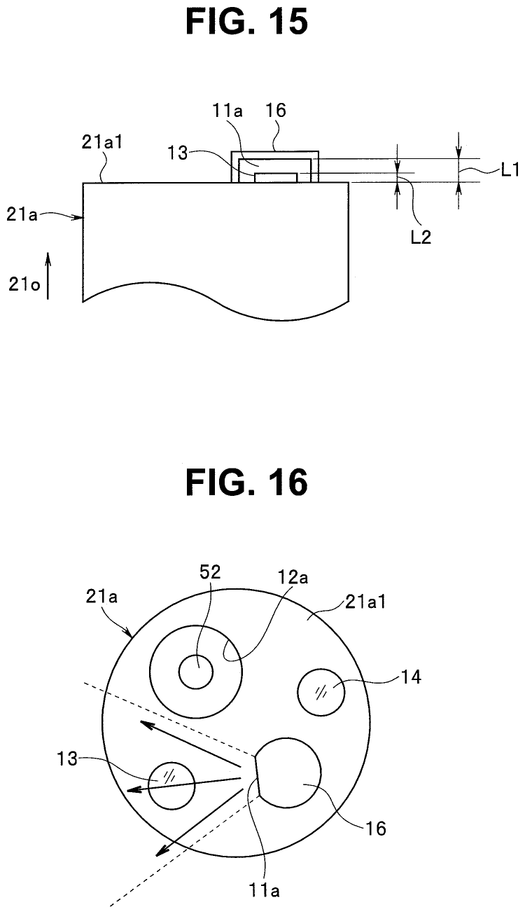

FIGS. 14 to 16 illustrate a third embodiment of the present disclosure and FIG. 14 is a sectional view illustrating a manner in which liquid flows from a liquid feeding channel 11 to an observation window 13 through a projecting shape portion 16. The projecting shape portion may include a projection. FIG. 15 is a diagram indicating a relationship between respective lengths of projection, in an axial direction 21o of an insertion portion 21, of a channel opening 11a of the liquid feeding channel 11 and the observation window 13. FIG. 16 is a diagram illustrating a distal end surface 21a1 of the distal end portion 21a of the endoscope 2, through which a laser probe 52 is inserted, together with flows of liquid. In the third embodiment, parts that are similar to the parts in the first and second embodiments are provided with reference numerals that are the same as the reference numerals in the first and second embodiments and description of such parts will appropriately be omitted and description will be provided mainly on differences.

As in the second embodiment, the present embodiment also employs a configuration in which feeding of liquid inside an endoscope 2 is performed not by a liquid feeding tube 63 but by a liquid feeding channel 11.

The liquid feeding channel 11 includes a projecting shape portion 16 that projects from a distal end surface 21a1 of a distal end portion 21a. In the present embodiment, a channel opening 11a is formed at a position, at which the channel opening 11a faces an observation window 13, in a side surface of the projecting shape portion 16. A part of the channel opening 11a functions as a first liquid feeding port OP1 and another part of the channel opening 11a functions as a second liquid feeding port OP2.

As illustrated in FIG. 15, the projecting shape portion 16 is configured in such a manner that a length L1 of projection, in an axial direction 21o of an insertion portion 21, of the channel opening 11a from the distal end surface 21a1 of the distal end portion 21a in the projecting shape portion 16 is longer than a length L2 of projection, in the axial direction 21o, of the observation window 13 from the distal end surface 21a1 of the distal end portion 21a (L1>L2).

The projecting shape portion 16 changes a direction of conveyance of liquid along the axial direction 21o of the insertion portion 21 by the liquid feeding channel 11 to a surface direction along the distal end surface 21a1 of the distal end portion 21a. Then, the channel opening 11a delivers a part of the liquid toward the observation window 13. Also, the channel opening 11a delivers another part of the liquid in a direction (direction of the inside of a subject) that is different from the direction of the observation window 13 as liquid for perfusion (see arrows in FIG. 16).

Such third embodiment as above exerts effects that are substantially similar to the effects of the above-described first and second embodiments and enables avoiding the distal end surface 21a1 from coming into contact with the inner wall of an organ as well as restricting a direction in which the liquid flows, because of the projecting shape portion 16 of the distal end portion 21a being provided and the channel opening 11a being made to face the observation window 13. Consequently, the observation window 13 is prevented from coming into contact with the inner wall of an organ and observation performance of the observation window 13 can be maintained.

Furthermore, since the length L1 of projection, in the axial direction 21o, of the channel opening 11a is made to be longer than the length L2, in the axial direction 21o, of the observation window 13, a flow of the liquid reliably covers the observation window 13, enabling preventing gas bubbles and a shattered stone from adhering to the observation window 13.

### Fourth Embodiment

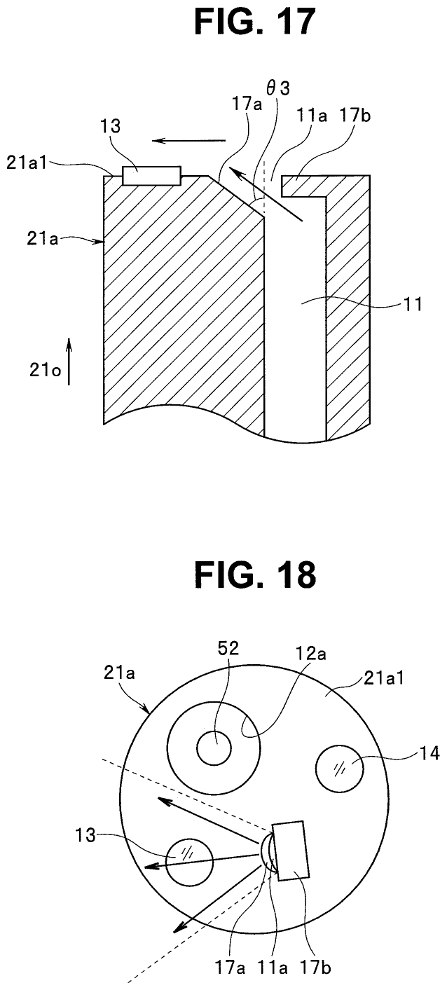

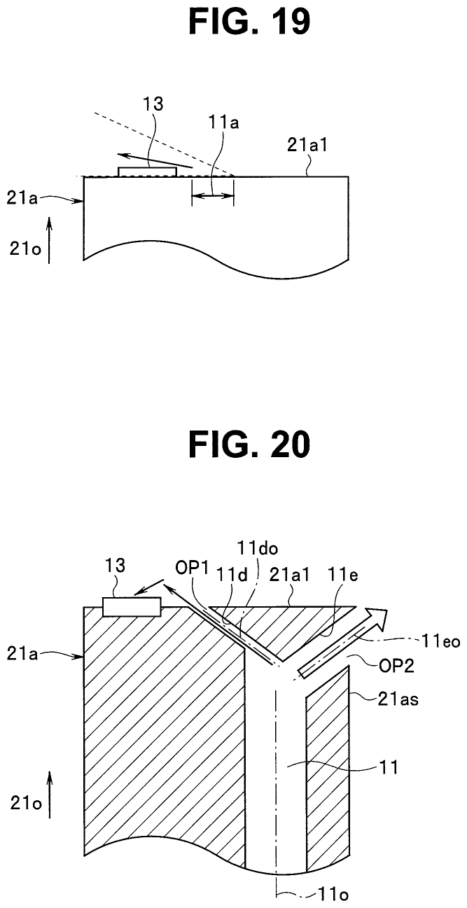

FIGS. 17 to 19 illustrate a fourth embodiment of the present disclosure and FIG. 17 is a sectional view illustrating a configuration of a part around a channel opening 11a of a liquid feeding channel 11. FIG. 18 is a diagram illustrating a distal end surface 21a1 of a distal end portion 21a of an endoscope 2, through which a laser probe 52 is inserted, together with flows of liquid. FIG. 19 is a diagram illustrating a manner in which liquid is delivered from the channel opening 11a of the liquid feeding channel 11 to an observation window 13 as viewed from a lateral side. In the fourth embodiment, parts that are similar to the parts in the first to third embodiments are provided with reference numerals that are the same as the reference numerals in the in the first to third embodiments and description of such parts will appropriately be omitted and description will be provided mainly on differences.

As in the second and third embodiments, the present embodiment employs a configuration in which feeding of liquid inside an endoscope 2 is performed not by a liquid feeding tube 63 but a liquid feeding channel 11.

The liquid feeding channel 11 includes a slanted surface 17a intersecting with an axial direction 21o of an insertion portion 21 and extending toward an observation window 13, in a distal end portion 21a. The slanted surface 17a is connected to, and ends at, a distal end surface 21a1 at a position close to the observation window 13. Also, an extending shape portion 17b that extends to the slanted surface 17a side is provided on the opposite side of the distal end side of the liquid feeding channel 11 from the slanted surface 17a. The extending shape portion 17b extends along the distal end surface 21a1 of the distal end portion 21a and does not project from the distal end surface 21a1 in the axial direction 21o.

The channel opening 11a is an opening between the slanted surface 17a, which ends at the distal end surface 21a1, and the extending shape portion 17b. In other words, the channel opening 11a is provided in the distal end surface 21a1 of the distal end portion 21a, the slanted surface 17a connecting to the distal end surface 21a1. A part of the channel opening 11a functions as a first liquid feeding port OP1 and another part of the channel opening 11a functions as a second liquid feeding port OP2.

The extending shape portion 17b changes a direction of conveyance of liquid along the axial direction 21o of the insertion portion 21 by the liquid feeding channel 11. The liquid subjected to the change of the direction of conveyance flows along the slanted surface 17a and is delivered from the channel opening 11a.

A part of the liquid delivered from the channel opening 11a serves as a liquid flow that covers the observation window 13 and a height of the liquid flow from the distal end surface 21a1 is higher than a height of the observation window 13. The liquid that has passed over the observation window 13 is further fed to the outside of the insertion portion 21.

Also, another part of the liquid delivered from the channel opening 11a flows in a direction (direction of the inside of a subject) that is different from the direction of the observation window 13 and serves as a liquid flow for perfusion (see arrows in FIG. 18).

An angle θ3 (see FIG. 17) of the slanted surface 17a to the axial direction 21o may be any appropriate angle as long as the angle enables a flow of the liquid delivered from the channel opening 11a to cover the observation window 13 and effectively remove gas bubbles and a shattered stone adhering to the observation window 13.

Such fourth embodiment as above exerts effects that are substantially similar to the effects of the above-described first to third embodiments and enables preventing deterioration in observation performance due to gas bubbles and/or a shattered stone by forming a liquid flow that covers the observation window 13 without making the extending shape portion 17b project in the axial direction 21o relative to the distal end surface 21a1, because of the slanted surface 17a that extends toward the observation window 13 being provided at the liquid feeding channel 11 in the distal end portion 21a.

In an ordinary endoscope, which is used, for example, in a gas, a liquid feeding channel that cleans an observation window basically projects from a distal end surface of an insertion portion. On the other hand, for example, a nephroscope to which the above-described endoscope 2 is applied is used in a liquid. Therefore, in the case of a structure in which the channel opening 11a is provided in a distal end surface 21a1 and does not project from a distal end surface 21a1 as in the present embodiment, even though liquid delivered from the channel opening 11a does not directly hit an observation window 13, the liquid creates a liquid flow by engulfing the surrounding liquid, enabling removal of gas bubbles on the observation window 13.

Also, in comparison with a structure in which a channel opening 11a is made to project from a distal end surface 21a1, a structure in which a channel opening 11a is provided in a distal end surface 21a1 is simple and thus is easy to manufacture. Furthermore, when there is no part that projects from the distal end surface 21a1, a length of the rigid distal end portion 21a does not become long, enhancing insertability of the insertion portion 21.

### Fifth Embodiment

FIG. 20 illustrates a fifth embodiment of the present disclosure and is a sectional view illustrating a configuration of a part around a channel opening 11a of a liquid feeding channel 11. In the fifth embodiment, parts that are similar to the parts in the first to fourth embodiments are provided with reference numerals that are the same as the reference numerals in the first to fourth embodiments and description of such parts will appropriately be omitted and description will be provided mainly on differences.

As in the second to fourth embodiments, the present embodiment employs a configuration in which feeding of liquid inside the endoscope 2 is performed not by a liquid feeding tube 63 but by a liquid feeding channel 11.

The liquid feeding channel 11 includes a first branch channel 11d and a second branch channel 11e in a distal end portion 21a.

The first branch channel 11d is provided in such a manner as to intersect with an axial direction 21o and extend toward an observation window 13. More specifically, a plane including a center line 11o of a part of the liquid feeding channel 11, the part being on the proximal end side relative to the first branch channel 11d and the second branch channel 11e, and a center line 11do of the first branch channel 11d intersects with the observation window 13. The first branch channel 11d communicates with a first liquid feeding port OP1 provided at a position in the distal end surface 21a1, the position being close to the observation window 13.

The second branch channel 11e is provided in such a manner as to intersect with the axial direction 21o in a direction that is different from a direction in which the first branch channel 11d intersects with the axial direction 21o and extend toward a side surface 21as of the distal end portion 21a. The second branch channel 11e communicates with a second liquid feeding port OP2, which is an opening provided in the side surface 21as of the distal end portion 21a.

In the example configuration illustrated in FIG. 20, a center line 11eo of the second branch channel 11e is further included in the plane including the center line 11o and the center line 11do of the first branch channel 11d. However, the configuration is a mere example and it is possible that the center line 11eo of the second branch channel 11e is not located in the plane including the center line 11o and the center line 11do of the first branch channel 11d (that is, it is possible that as the distal end surface 21a1 is viewed from the distal end side, the center line 11do and the center line 11eo are not on a line but form an angle).

The part of the liquid feeding channel 11, the part being on the proximal end side relative to the first branch channel 11d and the second branch channel 11e, conveys liquid in the axial direction 21o of the insertion portion 21. A part of the liquid conveyed in the axial direction 21o is conveyed by the first branch channel 11d and another part of the liquid is conveyed by the second branch channel 11e.

The liquid conveyed by the first branch channel 11d is delivered from the first liquid feeding port OP1 in a direction of the observation window 13. The liquid delivered by the first liquid feeding port OP1 removes a substance adhering to the observation window 13.

Also, the liquid conveyed by the second branch channel 11e is delivered from the second liquid feeding port OP2 in a direction (direction of the inside of a subject) that is different from the direction of the observation window 13. The inside of the subject is perfused by the liquid delivered by the second liquid feeding port OP2.

Such fifth embodiment as above exerts effects that are substantially similar to the effects of the above-described first to fourth embodiments and enables making a direction of a liquid flow toward the observation window 13 and a direction of a liquid flow for perfusion of the inside of a subject largely different from each other because of provision of the first branch channel 11d that communicates with the first liquid feeding port OP1 and the second branch channel 11e that communicates with the second liquid feeding port OP2.

Also, provision of the second liquid feeding port OP2 in the side surface 21as of the distal end portion 21a enables further reduction in flow of liquid, with which the inside of the subject has been perfused, toward the observation window 13.

The liquid is fed from the first liquid feeding port OP1 and the second liquid feeding port OP2, which are a plurality of liquid feeding ports, enabling reducing a flow rate of the liquid delivered from each one liquid feeding port and thus enabling prevention of scattering of a stone, which enhances collection efficiency.

In the above, an example in which laser light is applied from a laser probe to a stone inside a subject to shatter the stone has been described, but the present disclosure is not limited to this example and ultrasound may be applied from an ultrasound probe to a stone inside a subject to shatter the stone. Therefore, the probe may be either a laser probe or an ultrasound probe.

The above description has been provided mainly in terms of a case where the present disclosure provides an endoscope, an endoscope system and a stone collection method, but the present disclosure is not limited to this case, and may provide an actuation method for actuating an endoscope or an endoscope system in such a manner as described above or may provide, e.g., a computer program for making a computer perform processing that is similar to processing in an endoscope, an endoscope system or a stone collection method or a computer-readable non-transitory recording medium recording the computer program.

Also, the present disclosure is not limited to the above-described embodiments as they are, and in the practical phase, but the present disclosure can be embodied with components modified without departing from the gist of the disclosure. Also, various aspects of the disclosure can be formed by appropriate combinations of a plurality of components disclosed in the embodiments. For example, some components can be deleted from all of the components indicated in any of the embodiments. Furthermore, components in different embodiments may arbitrarily be combined. In this way, it should be understood that various modifications and applications are possible without departing from the gist of the disclosure.

What is claimed is:

1. An endoscope, comprising:
an insertion portion insertable into an inside of a subject;
an observation window provided in a distal end portion of the insertion portion;
a liquid feeding channel extending through the distal end portion of the insertion portion; and
at least one liquid feeding port,
wherein, in operation, liquid exits the at least one liquid feeding port, and
wherein liquid exiting the at least one liquid feeding port is directed in a first direction away from the observation window and toward the inside of the subject.

2. The endoscope according to claim 1, wherein the at least one liquid feeding port includes a first liquid feeding port portion and a second liquid feeding port portion,
wherein, in operation, liquid exiting the at least one liquid feeding port includes a first liquid portion that exits the first liquid feeding port portion and is directed in the first direction and a second liquid portion that exits the second liquid feeding port portion and is directed in a second direction toward the observation window.

3. The endoscope according to claim 2, wherein each of the first liquid feeding port and the second liquid feeding port is provided on a distal end side of the liquid feeding channel.

4. The endoscope according to claim 2, wherein the first liquid portion perfuses the inside of the subject and the second liquid portion removes a substance adhering to the observation window.

5. The endoscope according to claim 1, further comprising:
a liquid feeding tube inserted through an inside of the liquid feeding channel,
wherein the liquid feeding tube includes a tube distal end part projecting from an opening in a distal end surface of the distal end portion of the insertion portion,
wherein the tube distal end part includes a first opening formed in a side surface of the tube distal end part and a second opening formed in the side surface of the tube distal end part, the first opening separate from the second opening,
wherein the at least one liquid feeding port is the first opening,
wherein, in operation, the liquid feeding tube conveys liquid to the first opening and the second opening, and
wherein, in operation, liquid exiting the second opening is directed in a second direction toward the observation window.

6. The endoscope according to claim 5, wherein the liquid feeding tube includes a main channel, a first branch channel connecting the main channel to the first opening, and a second branch channel connecting the main channel to the second opening,
wherein a center axis of the second branch channel intersects a center axis of the main channel at a non-right angle, and
wherein the center axis of the second branch channel intersects a plane containing the distal end surface of the distal end portion of the insertion portion.

7. The endoscope according to claim 2, wherein the distal end portion includes a deflector that extends across a portion of the at least one liquid feeding port,
wherein, in operation, the liquid feeding channel conveys the liquid in an axial direction of the insertion portion and the second liquid portion that exits the second liquid feeding port portion contacts a surface of the deflector and is directed in the second direction, and
wherein the second direction is along a distal end surface of the distal end portion of the insertion portion.

8. The endoscope according to claim 7, wherein the deflector has a flat plate shape and the surface of the deflector forms an angle of more than 0 degrees and no more than 45 degrees with a plane containing the distal end surface of the distal end portion of the insertion portion,
wherein a normal to the surface of the deflector intersects a center line of the liquid feeding channel, and
wherein a plane including (i) the center line of the liquid feeding channel and (ii) the normal to the surface of the deflector intersects with the observation window.

9. The endoscope according to claim 2, wherein the liquid feeding channel includes an opening formed in a distal end surface of the distal end portion of the insertion portion.

10. The endoscope according to claim 9, wherein the distal end portion of the insertion portion includes a protrusion projecting from the distal end surface, wherein a first surface of the protrusion extends axially from the distal end surface and a second surface of the protrusion extends from the first surface across a center axis of the opening of the liquid feeding channel, and
wherein the at least one liquid feeding port is an opening formed in the first surface of protrusion.

11. The endoscope according to claim 10, wherein a distance in an axial direction of the insertion portion from the distal end surface to the second surface is a first distance,
wherein a distance in the axial direction of the insertion portion from the distal end surface to a distal end surface of the observation window is a second distance, and
wherein the first distance is greater than the second distance.

12. The endoscope according to claim 2, wherein the at least one liquid feeding port is located in a distal end surface of the distal end portion of the insertion portion,
wherein the at least one liquid feeding port is radially offset from a center axis of the liquid feeding channel, and
wherein at least a portion of a connection between the at least one liquid feeding port and the liquid feeding channel includes a surface oriented at an angle relative to the center axis of the liquid feeding channel and having one end connected to the distal end surface of the distal end portion of the insertion portion.

13. The endoscope according to claim 2, wherein the liquid feeding channel includes a first branch channel that communicates with the first liquid feeding port portion and a second branch channel that communicates with the second liquid feeding port portion.

14. The endoscope according to claim 13, wherein a first part of the liquid feeding channel is on a proximal end side relative to the first branch channel and the second branch channel,
wherein the first part of the liquid feeding channel extends in an axial direction of the insertion portion, and
wherein the second branch channel intersects the first part of the liquid feeding channel at a first location and extends toward the observation window.

15. The endoscope according to claim 14, wherein the first liquid feeding port portion is an opening provided in a side surface of the distal end portion,
wherein the first branch channel intersects the first part of the liquid feeding channel at a second location and extends toward the first liquid feeding port portion, and
wherein the first location is axially and radially different from the second location.

16. The endoscope according to claim 1, further comprising a treatment instrument channel extending through the insertion portion, the treatment instrument channel including a treatment instrument opening in the distal end portion,
wherein the treatment instrument channel is configured to slidably contain a probe configured to shatter a stone.

17. The endoscope according to claim 1, further comprising a suction channel inserted through the insertion portion,
wherein the suction channel is configured to suction up the liquid and stone from inside of the subject, and
wherein, in operation, the liquid is delivered from the at least one liquid feeding port and the inside of the subject is perfused by the liquid.

18. An endoscope system, comprising:
the endoscope according to claim 2; and
a liquid feeding/suction apparatus operably connected to the endoscope,
wherein the endoscope further includes a suction channel, and
wherein the liquid feeding/suction apparatus includes a liquid feeding pump configured to feed the liquid to the at least one liquid feeding port and a suction pump configured to suction up the liquid via the suction channel.

19. An endoscope system, comprising:
the endoscope according to claim 5; and
a liquid feeding/suction apparatus operably connected to the endoscope,
wherein the endoscope further includes a suction channel, and
wherein the liquid feeding/suction apparatus includes a liquid feeding pump configured to feed the liquid to the liquid feeding tube and a suction pump configured to suction up the liquid via the suction channel.

20. A stone collection method, comprising:
shattering a stone inside a subject via a probe;
conveying liquid via a liquid feeding channel of an endoscope;
delivering a first portion of the liquid via at least one liquid feeding port of the endoscope in a first direction toward an observation window provided in a distal end portion of an insertion portion of the endoscope;
delivering a second portion of the liquid via the at least one liquid feeding port of the endoscope in a second direction toward the inside of the subject from a second liquid feeding port of the liquid feeding channel; and
suctioning up the liquid and the shattered stone via a suction channel of the endoscope.
